(19) United States
(12) Patent Application Publication
(10) Pub. No.: US 2020/0229774 A1
(43) Pub. Date: Jul. 23, 2020

(54) Method to Quantify Hypertension, Aging Status and Vascular Properties in Vivo from Arterial Optical Plethysmograph Waveform Measurements

(71) Applicant: Grant Hocking, Alpharetta, GA (US)

(72) Inventor: Grant Hocking, Alpharetta, GA (US)

(21) Appl. No.: 16/744,931

(22) Filed: Jan. 16, 2020

Related U.S. Application Data
(60) Provisional application No. 62/793,591, filed on Jan. 17, 2019.

(51) Int. Cl. A61B 5/00; A61B 5/024; A61B 5/1455; A61B 5/026; A61B 5/0285; A61B 5/02

(52) U.S. Cl. CPC A61B 5/7278; A61B 5/02433; A61B 5/14552; A61B 5/0261; A61B 5/4836; A61B 5/6824; A61B 5/6826; A61B 5/0285; A61B 5/02007; A61B 5/7475; A61B 5/742

(57) ABSTRACT

The invention is an in vivo non-invasive method and apparatus for the measurement of hypertensive and aging status of a subject and the mechanical anelastic in vivo properties of arterial blood vessels. The method includes measuring a peripheral arterial pulse volume waveform (PVW) using an infra-red emitter and sensor positioned over an extremity and constructing the first time derivative, dPVW, of the PVW. From a ratio of the fall time over rise time of the dPVW and the time location of the second forward pulse wave, a hypertension index is derived. From the hypertensive index, the mechanical anelastic properties of peripheral arterial vascular vessels are determined. The change in the damping of the high frequency shear waves produces vasodilation/vasocontraction index which is a quantitative indicator of the extent of vasodilation, vasocontraction, or induced hypertension. From the index value the mechanical properties of arterial blood vessels are determined.

### CLAIM OF PRIORITY

This application claims priority from U.S. Provisional Patent Application Ser. No. 62/793, 591, filed Jan. 17, 2019, which is incorporated herein in its entirety.

### FIELD OF THE INVENTION

The present invention generally relates to the quantification of the hypertension and aging status of a living subject. More specifically, the present invention relates to systems and methods of using sensed peripheral arterial waveform measurements to assess hemodynamic parameters, such as hypertensive state, aging status, vasodilation or vasocontraction, and, also to quantify the mechanical anelastic properties of the blood vessels in vivo.

### BACKGROUND OF THE INVENTION

Conventional methods of establishing the hypertension state of a subject involves blood pressure measurements, and depending on the state of the subject's hypertension, medication may be prescribed to lower the subject's blood pressure. The effectiveness of such medication is monitored by blood pressure measurements. Provided the medication lowers the subject's blood pressure to acceptable levels, then it is presumed that the medication is considered effective in controlling the subject's hypertension. What impacts the prescribed medication has on the subject in general, and in particular the subject's blood vessels are unknown.

In subjects experiencing angina pectoris, glyceryl trinitrate may be prescribed as a vasodilator to inhibit the onset of angina pectoris during exercise. How effective this medication is to specific subjects is basically trial and error. During vasodilation, the blood vessels change their properties significantly, and without diagnostic measurements of these changes, the impact of the medication, and its potential impact on the subject's blood vessels is not known. Angina can also be due to narrowed or blocked arteries around the heart, ischemia, emotional stress, exposure to very hot or cold temperatures, heavy meals, and smoking.

The changes to the arterial vascular vessels mechanical properties from hypertension, aging, diabetes, mellitus, arteriosclerosis, hypercholesterolemia and ischemic heart disease are difficult to quantify, from simple pulse wave velocity (PWV) measurements, electrocardiogram (EKG) and blood pressure measurements. The anelastic in vivo properties of the peripheral arterial blood vessels can provide valuable insight into these processes on a subject's wellbeing, and the impact of medication to treat such disorders and their associated changes to the subject's arterial vascular vessel properties. The acute effect of vasoconstriction and vasodilation with resulting increase and decrease in blood pressure, have significant impact on the anelastic response of the body's peripheral arterial vascular vessels. In vivo quantification of these anelastic changes are essential in diagnosing the issues relating to aging and disease, and also as important, the impact of medication of changes to the peripheral arterial vascular vessels' behavior.

Arteries stiffen progressively with age and disease, even in the earliest stages of arteriosclerosis, prior to any clinical manifestation and anatomical evidence of the disease. In vivo quantification of minor changes in the peripheral artery blood vessels properties would provide an extremely useful clinical tool for the assessment of cardiovascular risk. In vivo quantification of minor changes in the peripheral artery blood vessels properties would provide an extremely useful clinical tool for the assessment of cardiovascular risk, from arterial vessel stiffening, plaque buildup, arteriosclerosis and/or elevated risk of aneurysm or dissection. In subjects suspected of sepsis knowing the subject's vasodilation/contraction state in real time would be a useful clinical tool to aid diagnosis. PWV and augmentation index are associated with cardiovascular burden, but do not have the sensitivity necessary to detect minor changes in the mechanical properties of the peripheral arterial blood vessels. Alternative methods for such an assessment are urgently needed.

### SUMMARY OF THE INVENTION

The present invention is an in vivo non-invasive method and apparatus for the measurement of the hypertensive and aging status of a subject, and the mechanical anelastic in vivo properties of the arterial blood vessels. The method requires measuring a peripheral arterial pulse volume waveform (PVW) by an optical plethysmograph, being an infra-red emitter and sensor positioned over a finger, as a clip, or ear or other extremity. Constructing from the peripheral arterial pulse volume waveform (PVW) its first time derivative (dPVW), and from a ratio of the fall over rise time of the first pulse flow rate waveform (dPVW) and the time location of the second forward pulse wave, the hypertensive and aging state of the subject can be quantified, and vasodilation or vasocontraction, and the mechanical anelastic properties of the subject's peripheral arterial vascular vessels can be assessed.

The current invention enables non-linear anelastic material properties of peripheral arterial blood vessels to be determined from a peripheral arterial pulse volume waveform (PVW) and from the first derivation of the PVW waveform, the rise and fall ratio of the first pulse wave is determined, and its ratio uniquely defines the Hypertensive Index (HI) and from this index the anelastic in vivo material properties of the arterial blood vessels can be quantified. Determining the fall to rise time ratio from the constructed dPVW waveform for any subject, the Hypertensive Index (HI) of that subject can be determined and its value will be equal to 0 for healthy normotensive subjects, but generally range from 0 to 100 for most subjects, and in cases of extreme hypertension can be >100. In some cases, the Hypertensive Index (HI) could be <0, for healthy subjects under extreme conditions such as exposure to temperature, altitude, and dehydration. The Hypertensive Index (HI) of a subject can be correlated to aging, and as such can determine whether elevated levels of the Hypertensive Index (HI) are related to the effects of aging, or are accelerated due to the impacts of disease, life style or medication on the respective subject.

The change in the damping of the high frequency shear waves is defined as Vasodilation/Vasocontraction Index (VI), which is a quantitative indicator of the extent of vasodilation, vasocontraction or induced hypertension. In this case, evaluation of the Index (VI) requires measurements prior to vasodilation, vasocontraction or induced hypertension to be precise in quantifying the degree of vasodilation or vasocontraction. The Index ((VI) is >0 for vasodilation and <0 for vasocontraction. Historical recoding of a subject's Index (VI) can enable the Index to be utilized with considerably greater accuracy.

Other objects, features and advantages of the present invention will become apparent upon reviewing the following description of the preferred embodiments of the invention, when taken in conjunction with the drawings and the claims.

### BRIEF DESCRIPTION OF THE DRAWINGS

FIG. 1 is a schematic isometric view of a subject's arm and associated graph illustrating a method embodying principles of the present invention, for quantifying the hypertension status of the subject and the in vivo anelastic properties of the arterial blood vessels.

FIG. 2 is a graph illustrating the averaged time history for forty (40) normotensive subjects of the peripheral arterial pulse volume waveform (PVW) recorded from an optical plethysmograph sensor positioned over a finger, and the time history of the constructed first time derivative of the PVW, and the averaged time history of the time shifted peripheral arterial pulse pressure waveform (PPW) recorded over the radial artery by a tonometer.

FIG. 3 is a graph illustrating the averaged time history for twenty (20) hypertensive subjects of the peripheral arterial pulse volume waveform (PVW) recorded from an optical plethysmograph sensor positioned over a finger, and the time history of the constructed first time derivative of the PVW, and the averaged time history of the time shifted peripheral arterial pulse pressure waveform (PPW) recorded over the radial artery by a tonometer.

FIG. 4 is a graph illustrating the normalized arterial pulse pressure plotted against the normalized arterial pulse volume as an average for the forty (40) normotensive subjects, and the thick wall three (3) component anelastic power law model.

FIG. 5 is a graph illustrating the normalized arterial pulse pressure plotted against the normalized arterial pulse volume as an average for the twenty (20) hypertensive subjects, and the thick wall three (3) component anelastic power law model.

FIG. 6 is a graph illustrating the time history for an elderly mildly hypertensive female subject of the peripheral arterial pulse volume waveform (PVW) recorded from an optical plethysmograph sensor positioned over a finger, and the time history of the constructed first time derivative of the PVW.

FIG. 7 is a graph illustrating the time history for an elderly mildly hypertensive male subject of the peripheral arterial pulse volume waveform (PVW) recorded from an optical plethysmograph sensor positioned over a finger, and the time history of the constructed first time derivative of the PVW.

FIG. 8 is a graph illustrating the time history for an elderly mildly hypertensive male subject of the peripheral arterial pulse volume waveform (PVW) recorded from an optical plethysmograph sensor positioned over the radial artery, and the time history of the constructed first time derivative of the PVW.

FIG. 9 is a graph illustrating a time history of the first time derivative of peripheral arterial pulse volume waveform (PVW) sensed from a finger, and the peripheral arterial pulse volume rate of change waveform (PAW) from a piezoelectric sensor positioned over the radial artery, and the second and first time derivatives of the respective waveforms, for quantification of the pulse wave velocity of the subject's arterial blood vessels, for quantification of the subject's hypertension and aging status, and the anelastic properties of the arterial blood vessels in vivo.

FIG. 10 is a graph illustrating the averaged normalized time history, for a subset of twenty (20) of the forty (40)) normotensive subjects following sublingually administration of 500 μg of glyceryl trinitrate (NTG), of the peripheral arterial pulse volume waveform (PVW) recorded from an optical plethysmograph sensor positioned over a finger, and the time history of the constructed first time derivative of the PVW, and the averaged time history of the time shifted peripheral arterial pulse pressure waveform (PPW) recorded over the radial artery by a tonometer.

FIG. 11 is a graph illustrating the normalized arterial pulse pressure plotted against the normalized arterial pulse volume as an average for the subset of twenty (20) normotensive subjects, following three (3) minutes after sublingually administration of 500 μg of glyceryl trinitrate (NTG), and the thick wall three (3) component anelastic power law model.

FIG. 12 is a graph illustrating a time history of the peripheral arterial waveform (dPVW) constructed from peripheral arterial pulse volume waveform (PVW), and the dPVW minus its two highest frequency intrinsic modes, and the recomposed conical wake highly dissipative shear waveform generated by the propagating arterial pulse, and the attenuation properties of these highly dissipative high frequency shear waveforms, for quantification of the hypertensive state of the subject and the in vivo anelastic properties of the arterial blood vessels.

### DETAILED DESCRIPTION OF THE DISCLOSED EMBODIMENT

Several embodiments of the present invention are described below and illustrated in the accompanying drawings. The present invention is an in vivo non-invasive method and apparatus for the measurement of the hypertensive state of a subject, and the mechanical anelastic in vivo properties of the arterial blood vessels. The method requires measuring a peripheral arterial pulse volume waveform (PVW), using an infra-red emitter and sensor positioned over a finger, as a clip, or ear or other extremity, being a transmitted sensed waveform, or as a reflective sensed peripheral arterial pulse volume waveform (PVW) by an infra-red emitter and sensor positioned over an artery, such as the radial artery. Constructing from the PVW waveform its first time derivative, and from a ratio of the rise and fall time of the first pulse flow rate, the hypertensive state of the subject can be quantified, and the mechanical anelastic properties of the subject's peripheral arterial vascular vessels can be determined. The PVW waveform can be transformed by either Fast Fourier Transform (FFT) or the power spectral density method to determine the respiratory and heart rates and associated higher frequencies.

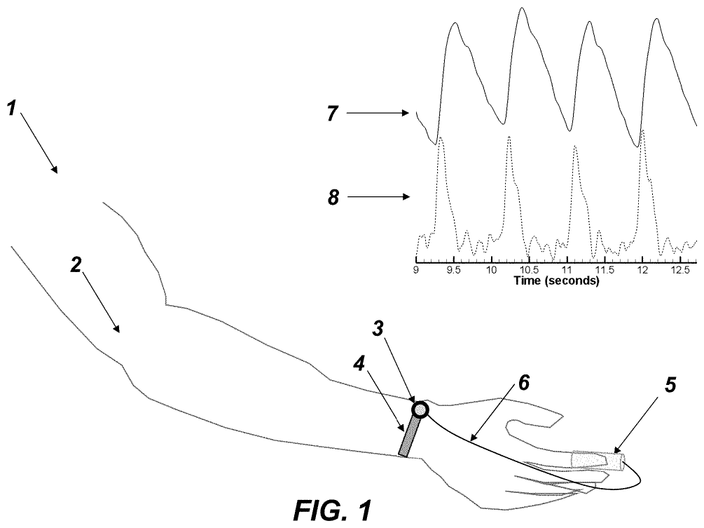

Representatively illustrated in FIG. 1 is a system 1 and associated method which embody principles of the present invention. The arm of the subject, 2, with a processing device 3 held in place by a strap 4, containing a reflective pulse optical plethysmograph sensor positioned over the subject's radial artery, and a piezoelectric sensor mounted on the optical plethysmograph sensor, with its axis normal to the transmitted light direction, for quantification of motion effects, with both sensors connected to the device 3. The pulse optical plethysmograph sensor 5 positioned over the finger of the subject and its associated piezoelectric motion sensor, are both connected to the processing device 3 by a lead denoted as 6. The measured peripheral arterial pulse optical plethysmograph waveform (PVW), and its constructed first time derivative (dPVW) are shown as time series 7, and 8 respectively. The constructed first time derivative (dPVW) of the PVW is calculated by the processing device 3, and the rise and fall times of the first forward pulse flow wave is determined from the dPVW by the device 3. The ratio of the first pulse wave fall time to its rise time, provides a direct measure of the subject's hypertension status, and can determined the mechanical anelastic properties of the subject's peripheral arterial vascular vessels, as described further in the other diagrams.

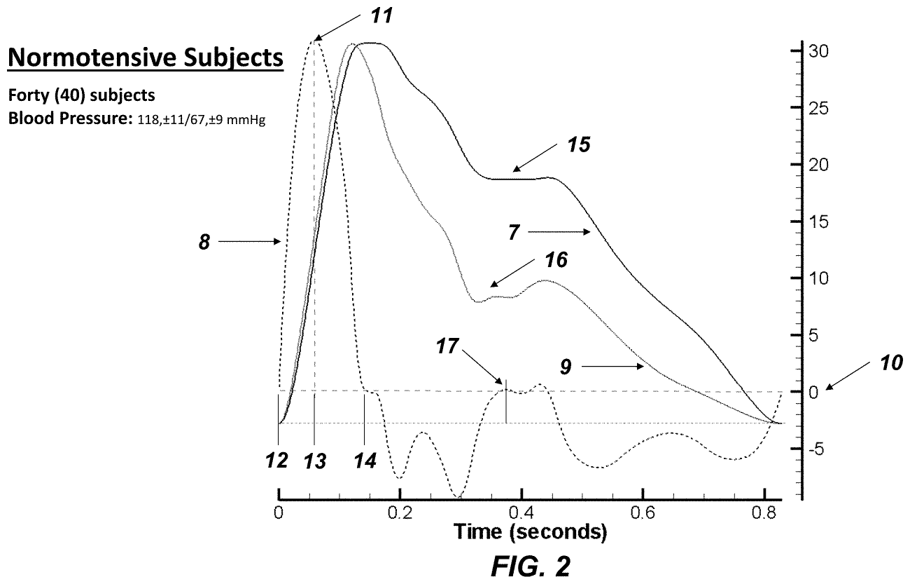

As depicted in FIG. 2, the graph illustrates the averaged normalized one heart cycle time history for forty (40) normotensive subjects peripheral arterial pulse optical plethysmograph waveform (PVW), denoted as 7, recorded from an optical plethysmograph sensor positioned over a finger, and the time history of the constructed first time derivative of the PVW being the dPVW, denoted as 8, and the averaged normalized time history of the time shifted peripheral arterial pulse pressure waveform (PPW) recorded over the radial artery by applanation tonometry by a piezo-resistive cantilever transducer, as denoted by 9. The measured waveforms, Millasseau et al., 2000, were normalized prior to being averaged for the forty (40) healthy normotensive subjects, aged from 24 to 80 years. All forty of the subjects had no previous history of hypertension or cardiovascular disease, and all were normotensive (office blood pressure <140/90 mm Hg), prior to the time of the study. Blood pressure measurements during the study were (mean, ±standard deviation) 118, ±11/67, ±9 mm Hg. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave.

The ratio of the fall time to the rise time of the first pulse wave for the normotensive subjects as determined from points 12, 13 and 14 is 1.8. The rise and fall times of the first and subsequent pulse waves are important and highly dependent on the peripheral arterial blood vessel mechanical anelastic properties. The pulse is a soliton and as such maintains its shape virtually unattenuated provided the energy lost by anelasticity is equivalent to the loss due to dispersion. When these losses are equal, the pulse wave travels as a soliton with no change in shape until it interacts with another forward or backward traveling pulse wave, and upon separation of the two interacting soliton waves, the waves have the same shape to that before the interaction, and there is only a time shift to distinguished that the two waves have undergone an interaction. The solution of the interaction of two solitons is not linear, and so requires a non-linear approach to differentiation between the various pulse waveform. If the energy lost by anelasticity of the peripheral blood vessels deviates from a Quality factor (defined later in equation (2)) of Q=3, then the shape (fall and rise times) of the first pulse wave will change, and it is this change that can be directly correlated to the peripheral arterial blood vessel anelastic properties. Alternatively, rather than determine the ratios at the zero ordinate of the dPVW, it could be determined at mid-height and thus remove the bias due to reflected waves have on the computed ratios. The second forward pulse wave is shown as 15 on the pulse volume waveform PVW, 7, and is also shown as 16 on the measured pulse pressure waveform, 9. The second forward pulse wave, which causes closure of the aortic valve, is shown as 17 on the dPVW waveform, and its arrival time position measured from the foot of the PVW in the heat beat cycle is 0.37 seconds.

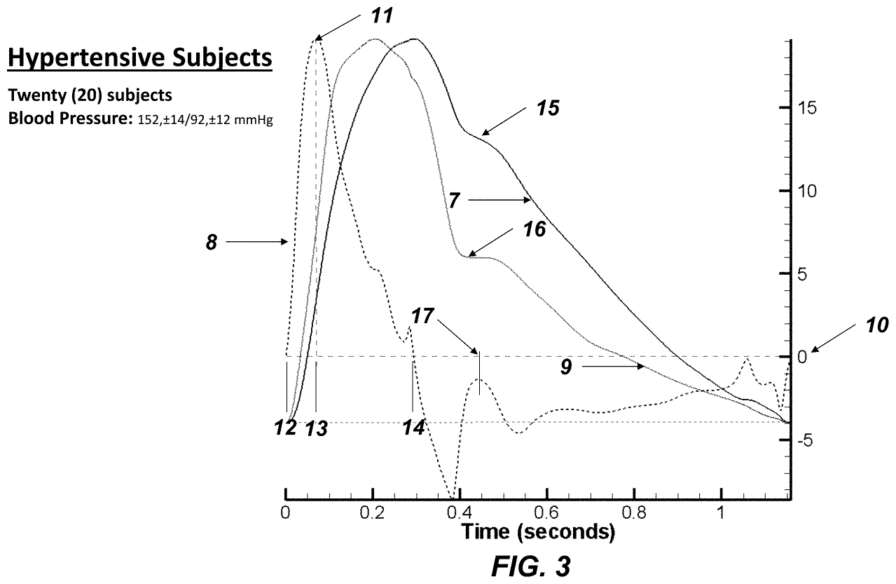

As depicted in FIG. 3, the graph illustrates the averaged normalized one heart cycle time history for twenty (20) hypertensive subjects, of their peripheral arterial pulse volume waveform (PVW), denoted as 7, recorded from an optical plethysmograph sensor positioned over a finger, and the time history of the constructed first time derivative of the PVW being the dPVW, denoted as 8, and the averaged normalized time history of the time shifted peripheral arterial pulse pressure waveform recorded over the radial artery by applanation tonometry by a piezo-resistive cantilever transducer (PPW), is denoted as 9. The measured waveforms, Millasseau et al., 2000, were normalized prior to being averaged for the twenty (20) hypertensive subjects, aged from 24 to 80 years. Hypertension was diagnosed on the basis of ≥3 measurements of office blood pressure >140/90 mm Hg, with each measurement separated by at least a week. None of the hypertensive subjects had clinical evidence of cardiovascular disease other than hypertension. Twelve (12) of the subjects were receiving antihypertensive therapy at the time of the study, (diuretics, 7 of 12; β-adrenoreceptor antagonists, 5 of 12; α-adrenoreceptor antagonists, 1 of 12; ACE inhibitors, 3 of 12; angiotensin II receptor antagonists, 2 of 12; and calcium channel blockers, 4 of 12). Blood pressure at the time of the study for the hypertensive subjects was 152, ±14/92±12 mm Hg. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave.

The ratio of the fall time to the rise time of the first pulse wave for the normotensive subjects as determined from points 12, 13 and 14 is 3.4, a significant difference from the ratio determined for the normotensive subjects, which was 1.8. Normalizing the fall to rise time ratio to the normotensive subjects, the normalized fall to rise time for the hypertensive subjects is 1.9, and by construction of a Hypertensive Index (HI) from the forty (40) normotensive subjects as a HI=0, and the twenty (20) hypertensive subjects having a HI=100. Determining the fall to rise time ratio from the constructed dPVW waveform for any subject, the Hypertensive Index (HI) of that subject can be determined and its value will be equal to 0 for healthy normotensive subjects, but generally range from 0 to 100 for most subjects, and in cases of extreme hypertension can be >100. Alternatively, rather than determine the ratios at the zero ordinate of the dPVW, it could be determined at mid-height and thus remove the bias due to reflected waves have on the computed ratios. In some cases, the Hypertensive Index (HI) could be <0, for healthy subjects under extreme conditions such as exposure to temperature, altitude, and dehydration. The Hypertensive Index (HI) of a subject can be correlated to age, and as such can determine whether elevated levels of the Hypertensive Index (HI) are related to the effects of aging, or being accelerated due to the impacts of disease, life style or medication on the respective subject. The second forward pulse wave is shown as 15 on the pulse volume waveform PVW, 7, and is also shown as 16 on the measured pulse pressure waveform, 9. The second forward pulse wave, which causes closure of the aortic valve, is shown as 17 on the dPVW waveform, and its arrival time location from the foot of the PVW in the heart beat cycle is 0.45 seconds. The arrival time location of the second forward pulse wave from the normotensive subjects to the hypertensive subjects is attributed solely to hypertension, which is not considered to be aging related hypertension. The arrival time of the second forward pulse wave was 0.37 seconds for the normotensive subjects. The later arrival time of the second forward wave for the hypertensive subjects is not due to aging, but either a genetically predisposition to hypertension, or related to disease or life style impacts.

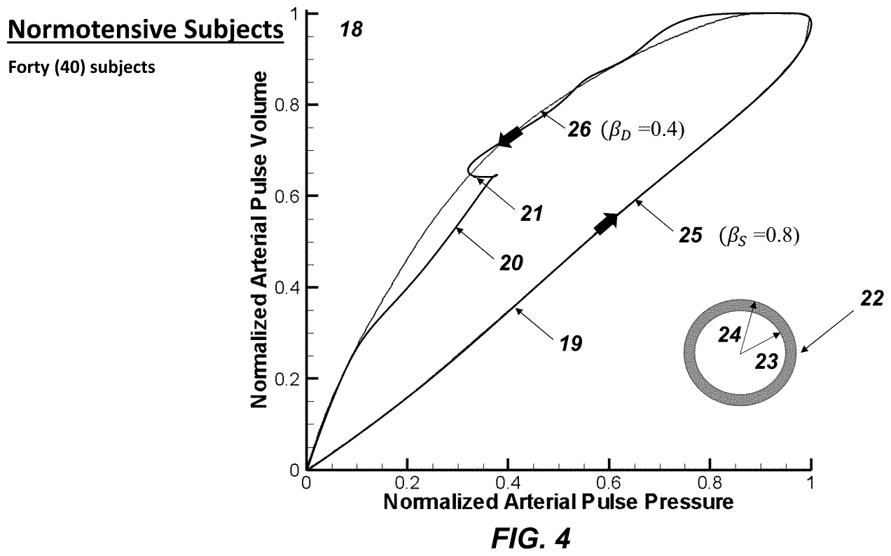

As depicted in FIG. 4, the graph illustrates the normalized arterial pulse pressure versus normalized arterial pulse volume for the forty (40) normotensive subjects, denoted as 18, constructed from the PVW and PPW waveforms, denoted earlier as 7 and 9 respectively. The rise (pressurizing) portion of the pulse pressure versus pulse volume is shown as 19, and the fall (depressurizing) portion is denoted as 20. Note that the fall portion 20 of the plot experiences load/unload cycles as denoted by 21.

As depicted in FIG. 4, and shown as 21, the graph illustrates the three (3) component thick wall anelastic power law model denoted as 22, with inner wall radius 23 and outer wall radius 24, fitted to the normalized arterial pulse pressure versus normalized arterial pulse volume for the forty (40) normotensive subjects. The anelastic power law model is an analytical closed form solution of an incompressible material described by equation (1) for the systolic, pressurizing (loading) path, with a similar equation for the diastolic, depressurizing (unloading) path. The anelastic model has a power law coefficient for the systolic portion, βs and the diastolic portion PD.

$$( \frac{δ A}{A} ) = ( \frac{{β}_{S} Δ P}{{G}_{R} ( 1 - {( \frac{a}{b} )}^{2 {β}_{S}} )} ) [ 1 - {( \frac{Δ P - P}{Δ P} )}^{{β}_{S}} ] \qquad ( 1 )$$

where (δA/A) is the change in area over original area at a pulse pressure of P. ΔP is systolic minus diastolic pressure, G_{R} is the radial secant shear modulus, β_{S} is a power law coefficient for the systolic, i.e. loading (pressurizing) path, a is the inner wall radius, b is the outer wall radius, and β_{D} is a power law coefficient for the diastolic, i.e. depressurizing (unloading) path. For a β_{S}=1, the model is linear elastic, for β_{S}<1, the model softens with increasing pressure, and for β_{S}>1, the model stiffens with increasing pressure. The simple anelastic power law model has been used to model arteries, both large and small, the aorta, the arterioles and veins. The small and large arteries have similar power law coefficients of β_{S}<1 at rest and β_{S}>1 when vasodilated, while the aorta is much different having β_{S}>1, as do the arterioles.

The normalized arterial pulse pressure (P) versus normalized arterial pulse volume, being the change in area over original area, i.e. (δA/A) of the three component thick wall anelastic power law model fitted to the normotensive subjects data, is shown in FIG. 4. The rise (pressurizing) portion of the pulse pressure versus pulse volume for the power law model fitted to the measured data, is shown as 25, with a power law model value of β_{S}=0.8, and the purely fall (depressurizing) portion is denoted as 26, with a power law model value of β_{D}=0.4. As the arterial blood vessels are anelastic, they experience small load/unload cycles as the various pulse waves of the waveform arrive, as denoted by 21. The anelasticity of the model is given by the Quality factor, Q, which is the inverse of the energy lost divided by the total energy over a complete load/unload cycle. The Quality factor is related to the power law loading and unloading coefficients as given by equation (2).

$${Q}^{- 1} = \frac{1 - {β}_{S} {β}_{D}}{1 + 2 {β}_{D} + {β}_{S} {β}_{D}} \qquad ( 2 )$$

The area between the load/unload paths 25 and 26 is the energy lost during a complete load/unload cycle. For a β of 1 the model is linear elastic and thus Q tends to infinity, i.e. zero energy loss. The Quality factor, Q, for the fitted model shown in FIG. 4 is equal to 3.1, being considered the expected value of healthy arterial vascular blood vessels in vivo.

The blood vessels are composed of collagen (endothelium), elastin, smooth muscles and connective tissue. The arteries and veins differ significantly in their anelasticity, due to their significant different functions and applied loads. In the arteries, the collagen, elastin and smooth muscle have values of shear modulus in descending order of ˜10⁷ to 10⁶, and 10⁵ and 10⁴ Nm ⁻², respectively. The arterial elastic lamellae and smooth muscle cells are wrapped by a network of collagenous fibrils. Most of the collagen fibers are orientated circumferentially, but with some orientated obliquely and others longitudinally. Elastin and collagen fibers contribute to the artery's elasticity. In human beings, the number of elastic lamella is related to the anatomic location of the artery; muscular arteries have only one internal and external elastic lamina, while in the aorta there are some 60-90 elastic lamina. The number of elastic lamina decreases gradually towards the periphery of the arterial system. Arterial wall viscosity plays a major role in regulating the mechanical behavior of muscular arteries to their applied loads. The smooth muscle component of the artery wall is considered an important element of the artery that contributes to its viscosity. All components of the artery wall may contribute to its viscosity, but the smooth muscle is the only component to respond to physiological stimulus. Furthermore, these components are influenced both by physiological and pathological changes in the mucopolysaccharide, in which they are embedded. The model could be made more complex with differing layers in the blood vessel wall, anisotropic properties, and also include time dependent effects. However, with that complexity the unique quantification to define the model parameters from non-invasive in vivo measurements becomes unwieldy, so a simple model that contains the essential behavior of the blood vessels' anelastic compliance is preferred. Therefore, the three component model described here is considered a suitable choice; however, the method is not limited to this model's simplicity nor limited to a three component anelastic model, as a fourth component can be added to account for quantifying the effects of arterial vessels' axial tethering in vivo.

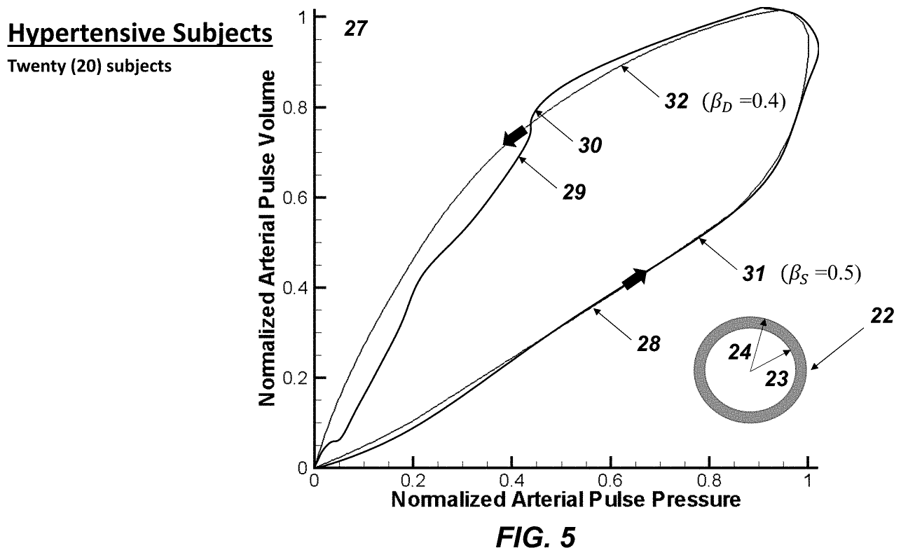

As depicted in FIG. 5, the graph illustrates the normalized time shifted arterial pulse pressure versus the normalized arterial pulse volume for the twenty (20) hypertensive subjects, denoted as 27, constructed from the PVW and PPW waveforms, denoted earlier as 7 and 9 respectively. The rise (pressurizing) portion of the pulse pressure versus pulse volume is shown as 28, and the fall (depressurizing) portion is denoted as 29. As the arterial blood vessels are anelastic, they experience small load/unload cycles as the various pulse waves of the waveform arrive, as denoted by 30. The three (3) component thick wall anelastic power law model denoted as 22, with inner wall radius 23 and outer wall radius 24, is fitted to the normalized arterial pulse pressure versus normalized arterial pulse volume for the twenty (20) hypertensive subjects. The rise (pressurizing) portion of the pulse pressure versus pulse volume for the power law model fitted to the measured data, is shown as 31, with a power law model value of β_{S}=0.5, and the purely fall (depressurizing) portion is denoted as 32, with a power law model value of β_{D}=0.4. The Quality factor, Q, for the fitted model shown as 27 in FIG. 5 is Q=2.5, which translates to a 40% energy loss over a complete load/unload cycle.

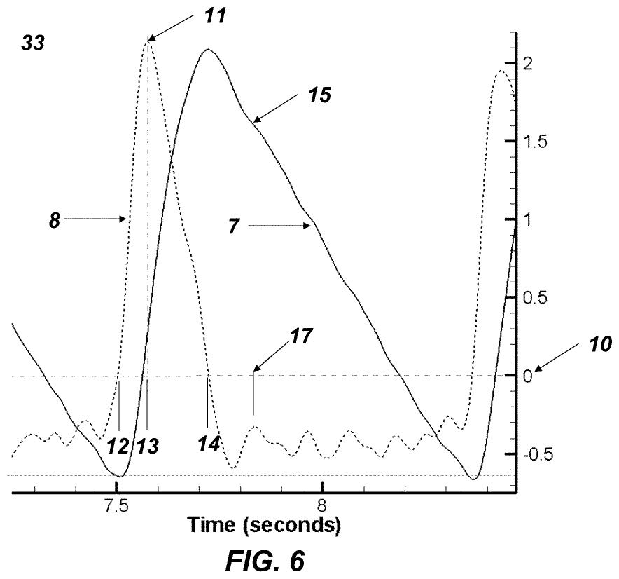

As depicted in FIG. 6, the graph illustrates the normalized one heart cycle time history 33 for an elderly female subject of 72 years old, with moderate hypertension, having a blood pressure of 137/87 mm Hg, and receiving antihypertensive therapy of β-adrenoreceptor antagonists. The peripheral arterial pulse volume waveform (PVW), denoted as 7, recorded from an optical plethysmograph sensor positioned over a finger, and the time history of the constructed first time derivative of the PVW being the dPVW, denoted as 8. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave. The ratio of the fall time to the rise time of the first pulse wave for the normotensive subjects as determined from points 12, 13 and 14 is 2.8, and normalizing this fall to rise time ratio to the forty (40) normotensive subjects, gives a normalized ratio of 1.55, which lies approximately midway between the forty (40) normotensive subjects and the twenty (20) hypertensive subjects. The second forward pulse wave is poorly seen, if at all, as shown as 15 on the pulse volume waveform PVW, 7, and is also shown as 16 on the measured pulse pressure waveform, 9. The second forward pulse wave, which causes closure of the aortic valve, is clearly discernable, but faint, and is as shown as 17 on the dPVW waveform, 8. The arrival time position for the second forward pulse wave measured from the foot of the PVW was determined to be 0.43 seconds, so the arrival time of the second forward wave is similar to that of the twenty (20) hypertensive subjects, which was 0.45 seconds, and thus indicates that this person hypertension is not age related.

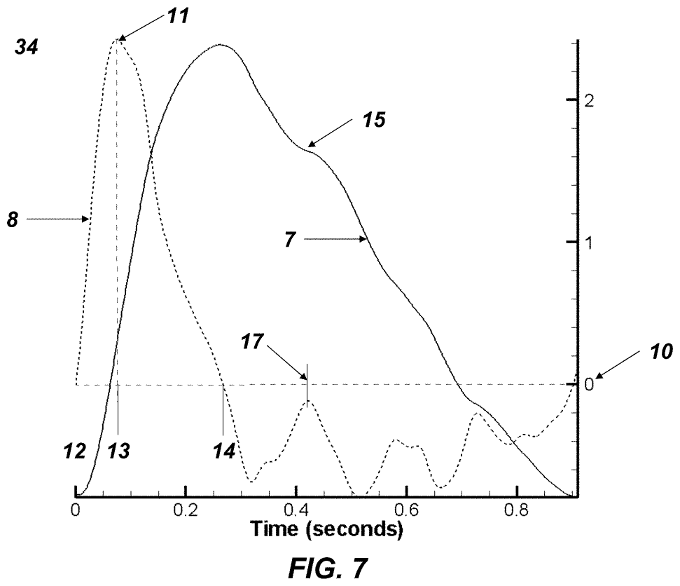

The normalized fall to rise time ratio is a direct measure of the hypertensive status of a subject, and from the peripheral arterial pulse optical plethysmograph waveform (PVW) measurements, the hypertensive status is determined. In the case of the subject shown in FIG. 6, the normalized ratio is 1.55, lying between a value of 1.0 for the forty (40) normotensive subjects and 1.9 for the twenty (20) hypertensive subjects, resulting in a Hypertensive Index (HI) magnitude for this subject of 61. The arrival time location of the second forward pulse wave, quantifies the hypertension for this subject is considered not age related. From the HI magnitude, the anelastic power law model parameters can be determined, assuming a linear change from a normalized ratio from 0 to 100, for the normotensive and hypertensive subjects respectively. Then, for the subject in FIG. 7, the rise (pressurizing) value is β_{S}=0.55, and the fall (depressurizing) portion is given as β_{D}=0.4, for a Quality factor, of Q=2.6.

As depicted in FIG. 7, the graph illustrates the normalized one heart cycle time history 34 for an elderly male subject of 69 years old, with mild to moderate hypertension, having a blood pressure of 120/78 mm Hg, and receiving antihypertensive therapy of angiotensin II receptor antagonists, only since the age of 65. The peripheral arterial pulse volume waveform (PVW), denoted as 7, recorded from an optical plethysmograph sensor positioned over a finger, and the time history of the first time derivative of the PVW being the dPVW, denoted as 8. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave. The ratio of the fall time to the rise time of the first pulse wave for this subject was determined from points 12, 13 and 14 is 2.5, and normalizing this fall to rise time ratio to the forty (40) normotensive subjects, gives a normalized ratio of 1.4, which lies below the midway normalized fall to rise time ratio value, between the forty (40) normotensive subjects and the twenty (20) hypertensive subjects. The second forward pulse wave can be seen, as shown as 15 on the pulse volume waveform PVW, 7. The second forward pulse wave, which causes closure of the aortic valve, is clearly discernable as shown as 17 on the dPVW waveform, 8. The arrival time location of the second forward pulse wave measured from the foot of the PVW is 0.36 seconds, and is similar to the arrival time for the forty (20) normotensive subjects, and as such the hypertension of this subject is determined to be solely age related.

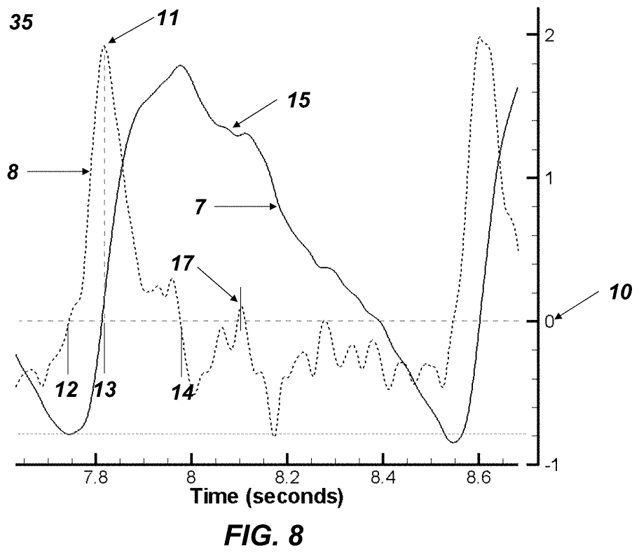

As depicted in FIG. 8, the graph illustrates the normalized one heart cycle time history 35 for the same male subject of 69 years old, with mild to moderate hypertension, as shown as 34 in FIG. 7. The peripheral arterial pulse volume waveform (PVW), denoted as 7, recorded from an optical plethysmograph sensor positioned over the radial artery, and the time history of the first time derivative of the PVW being the dPVW, denoted as 8. Note the significant number of reflections measured at the radial artery, compared to that measured over the finger, 34, for the same subject. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave. The ratio of the fall time to the rise time of the first pulse wave for the normotensive subjects as determined from points 12, 13 and 14 is 2.5, and normalizing this fall to rise time ratio to the forty (40) normotensive subjects, gives a normalized ratio of 1.4, which lies below the midway normalized fall to rise time ratio value, between the forty (40) normotensive subjects and the twenty (20) hypertensive subjects. The second forward pulse wave can be seen, as shown as 15 on the pulse volume waveform PVW, 7. The second forward pulse wave, which causes closure of the aortic valve, is discernable, even due to the numerous reflections contained in the PVW and dPVW waveforms at this location, and is shown as 17 on the dPVW waveform, 8.

The normalized fall to rise time ratio is a direct measure of the hypertensive status of a subject, and from only peripheral arterial pulse volume waveform (PVW) measurements, the hypertensive status of a subject can be quantified. In the case of the subject shown in FIGS. 7 and 8, for peripheral arterial pulse volume waveform (PVW) in measurements conducted on the finger and over the radial artery, respectively. The normalized ratio was 1.39, lying between a value of 1.0 for the forty (40) normotensive subjects and 1.9 for the twenty (20) hypertensive subjects, resulting in a Hypertensive Index (HI) magnitude for this subject of 43. The arrival time location of the second forward pulse wave as measured from the foot of the PVW for this subject is the same as the forty (40) normotensive subjects, so the hypertension of this subject is determined to be solely age related. The aging vector of the Hypertension Index for this subject is one. Thus, the Hypertension Index (HI) of this subject is determined to be HI=43 being its magnitude and that from the second forward wave arrival time, determines that the hypertension for this subject is considered totally aging related.

From the HI magnitude, the anelastic power law model parameters can be determined, assuming a linear change from a normalized ratio from 0 to 100, for the normotensive and hypertensive subjects respectively. Thus, for the subject in FIGS. 7 and 8, the rise (pressurizing) values are β_{S}=0.67, and the fall (depressurizing) portion is β_{D}=0.4, for a Quality factor, of Q=2.8.

From the peripheral arterial pulse volume waveform (PVW) measurements and simultaneous measurement of the peripheral pulse pressure waveform (PPW) over the subject's radial artery, by a force sensor tonometer, the magnitude of the out of phase of the PPW waveform, which leads the PVW waveform, and the plot of time shifted (to account for the out of phase) pulse pressure versus pulse volume, the β values of the subject's radial artery were exactly β_{S}=0.67 and β_{D}=0.4 as similarly determined above by the fall to rise ratio, yielding the HI magnitude, and using linear interpretation from the normotensive to hypertensive subject database. Assuming a linear relationship between hypertrophy and the systolic power law coefficient, the a/b ratio of the mildly hypertensive 69 year old male subject is 0.785, from data given by Laurent et al., 1994, of a/b=0.81 and 0.75 for the normotensive and hypertensive subjects, respectively.

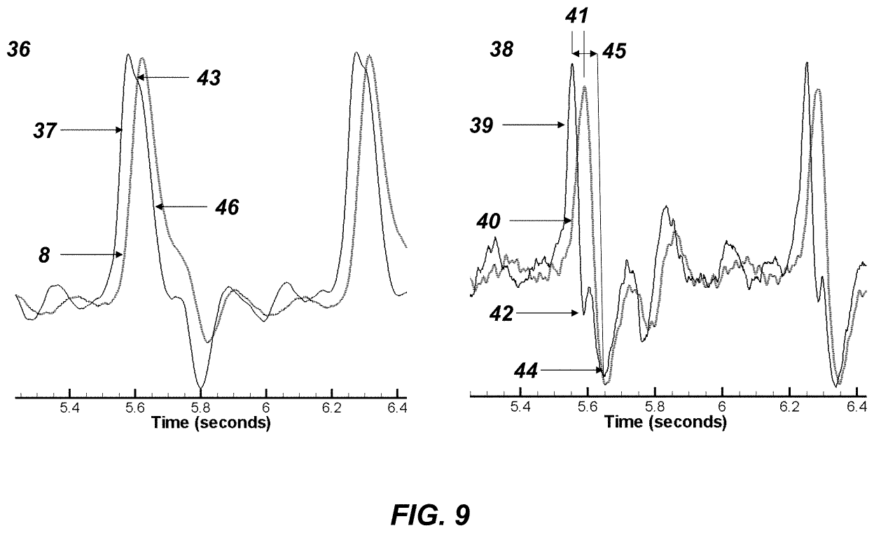

As depicted in FIG. 9 and shown as 36, the graph illustrates the measured peripheral arterial pulse volume rate of change waveform (PAW) from a piezoelectric sensor positioned over the radial artery and contained in the wrist band 4. The PAW response with time is denoted as 37, and the constructed first time derivative (dPVW) 8, of the peripheral arterial pulse volume waveform (PVW) measured at the finger by a sensor 5, is shown as its response with time as 8. These measured data were obtained for the same male subject of 69 years old, with mild to moderate hypertension, as shown as 34 in FIGS. 7 and 35 in FIG. 8 for the optical plethysmograph sensor positioned over the finger and radial artery, respectively. As shown in 38, are the time derivatives of both the PAW and dPVW shown as 39 and 40 respectively. The travel time for the arterial pulse to travel from the radial artery to the finger clip sensor is best determined from the derivative plots given in 38, and is denoted as 41. The pulse wave velocity of the subject is given by the distance from the radial artery to the pulse optical plethysmograph finger sensor 5 divided by the travel time 41. The first reflected backward arterial wave experienced by the piezoelectric sensor denoted as 42 occurs as shown in 38, and is seen as 43, resulting in the slope change noted as 36. The second reflected backward arterial wave is from the arterioles in the finger and occurs at 44, resulting in a two way travel time of 45, being twice the travel time given by 41, and yields the change in slope of 37 as shown by 46. The distance between the finger sensor and radial artery for this subject was 18 cm and the single way travel time 41 was 0.035seconds, with a double way travel time 45 of 0.07seconds, yielding an arterial pulse wave velocity for this subject of 5.1 m/s.

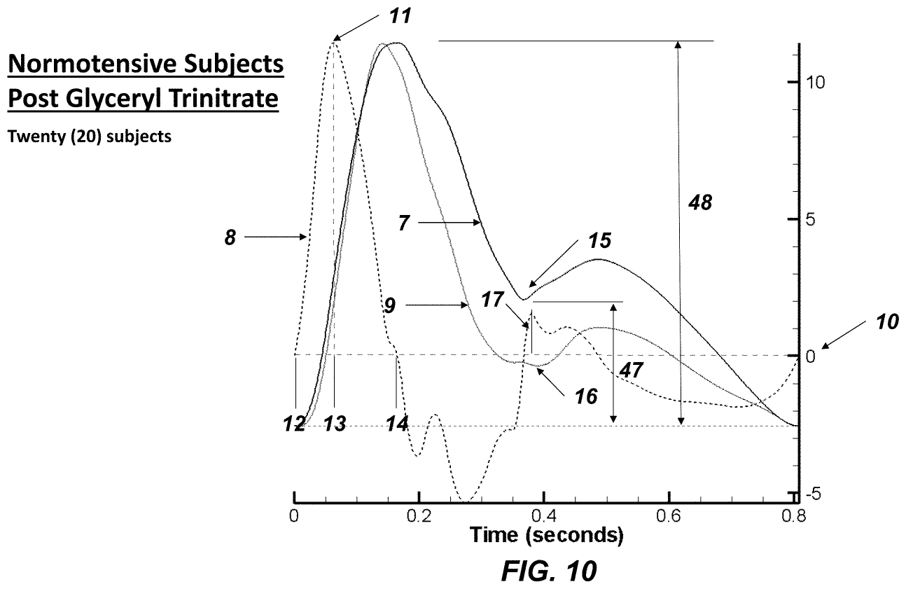

As depicted in FIG. 10, the graph illustrates the averaged normalized one heart cycle time history for a subset of twelve (12) of the twenty (20)) normotensive subjects following sublingually administration of 500 μg of glyceryl trinitrate (NTG). The peripheral arterial pulse volume waveform (PVW), denoted as 7, recorded from an optical plethysmograph sensor positioned over a finger, and the time history of the constructed first time derivative of the PVW being the dPVW, denoted as 8, and the averaged normalized time history of the time shifted peripheral arterial pulse pressure waveform (PPW) recorded over the radial artery by applanation tonometry by a piezo-resistive cantilever transducer, is denoted as 9. The waveforms were recorded 3 minutes after the NTG was administered, which is when the effects of the NTG are at a maximum. The zero ordinate of the dPVW constructed waveform is shown as 10. The first pulse wave peak is denoted as 11. The rise and fall time intervals of the first pulse wave are given by the difference in the time abscissa of points denoted as 12, 13 and 14. With the points, being the intersection of the zero ordinate 10 and the constructed dPVW waveform, point 12 being the start of the rise of the first pulse wave, point 13 being the maximum of the first pulse wave, and point 14 being the end of the fall of the first pulse wave. The ratio of the fall time to the rise time of the first pulse wave for the normotensive subjects as determined from points 12, 13 and 14 is 1.8, which is the same as the forty (40) normotensive subjects prior to any NTG being administered. That is, the NTG had no discernable effect on this fall to rise time ratio of the first pulse wave. The second forward pulse wave is shown as 15 on the pulse volume waveform PVW, 7, and is also shown as 16 on the measured pulse pressure waveform, 9. The second forward pulse wave, which causes closure of the aortic valve, is shown as 17 on the dPVW waveform. The second forward pulse wave arrival time location as measured from the foot of the PVW is 0.37 seconds, which is the same as the forty (40) normotensive subjects prior to any NTG being administered.

Note the significant differences in the second forward pulse wave in FIG. 10, i.e. with NTG taken effect, compared to that given in FIG. 2 for the subjects prior to any NTG being administered. The second forward pulse wave in FIG. 2 is 0.65 of the maximum pulse volume, and in FIG. 10 it is 0.31, denoted as the ratio of 47 to 48, and in this case being a percentage drop of 48% from the forty (40) normotensive subjects to the twenty (20) subset normotensive subjects following NTG administration. Similarly, the pulse pressure drops significantly, from 0.31 in FIG. 2, prior to NTG being administered, to 0.16, after NTG, as shown in FIG. 10, for the normotensive subjects prior and after NTG being administered. The ratio of the normalized pulse volume decline or rise, is a quantitative indicator of the extent of vasodilation or vasocontraction, and is given by VI, the Vasodilation/Vasocontraction Index. In this case, normalizing the pulse volume drop of 48% to an index value of 100, then the administration of 500 μg of NTG, resulted in a Vasodilation/Vasocontraction Index value of VI=100. In the case of vasocontraction, the index VI is a negative value. Determining the fall or rise of the normalized pulse volume ratio from the PVW waveform measured over the finger for quantifying the index (VI) can be difficult to detect, especially in aged subjects or subjects suffering from arteriosclerosis or hypertension. Alternatively, the PVW waveform measured over the radial artery, as shown in FIG. 7 compared to FIG. 8, can provide a more accurate measure of the change in pulse volume, due to either vasodilation or vasocontraction, and so this ratio can, in some cases, be better measured over the radial artery. Care needs to be taken with the reflections in the PVW waveform and its derivatives at this location. Alternatively, a piezoelectric sensor placed over an artery can better detect both the time location of the second forward pulse wave, and by integrating the piezoelectric sensor in the vicinity of the second forward pulse wave time location, the pulse volume change can be better determined for aged subjects or subjects suffering from arteriosclerosis or hypertension. The rate of pulse volume change in the vicinity of the second forward pulse wave can be determine over time and raise alerts if this time rate of change of pulse volume starts to accelerate.

In the above cases, for the assessment of the vasodilation/vasocontraction index VI, the first time derivative of the PVW waveform, being defined as the dPVW waveform, can be reconstructed by the empirical mode decomposition method (EMD) for a better evaluation of the vasodilation/vasocontraction index VI. The dPVW waveform can be decomposed into its intrinsic oscillatory modes, being typically fourteen (14) intrinsic oscillatory modes decomposed from the dPVW waveform 8, as earlier disclosed and detailed in U.S. Pat. No. 5,983,162, and named as the empirical mode decomposition (EMD) method, and the method further refined and known as the ensemble empirical mode decomposition (EEMD) method, collectively denoted here as the EMD method. The decomposition of the dPVW waveform into its intrinsic oscillatory modes, begins with the shortest period oscillatory mode first being quantified, that mode then subtracted from the original dPVW waveform, and the next shortest period oscillatory mode is found, and so on, until all the intrinsic oscillatory modes are determined. The sum of all of the intrinsic oscillatory modes yields the original dPVW waveform 8. The intrinsic oscillatory modes are general in nature and can accommodate non-linear waveform analysis, and unlike constant amplitude and/or frequency in a simple harmonic component, the intrinsic oscillatory modes can have variable amplitude and frequency along the time axis. In this case, a reconstructed PVW waveform can be found from the intrinsic oscillatory modes of the dPVW waveform, to better determine the vasodilation/vasocontraction index VI.

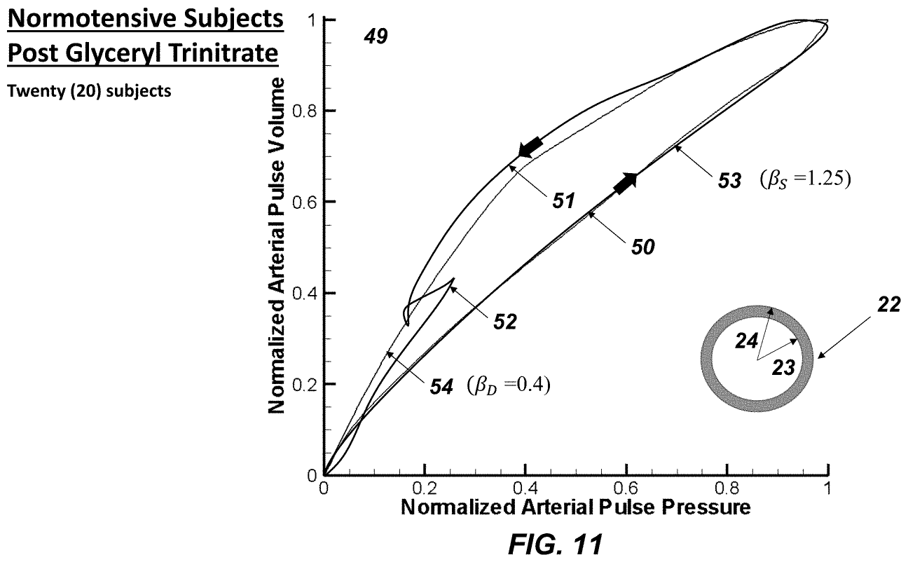

As depicted in FIG. 11, the graph illustrates the normalized arterial pulse pressure versus normalized arterial pulse volume for the twenty (20) subset of the forty (40) normotensive subjects, following three (3) minutes after NTG being administered, denoted as 49, constructed from the PVW and PPW waveforms, denoted earlier as 7 and 9 respectively. The rise (pressurizing) portion of the pulse pressure versus pulse volume is shown as 50, and the fall (depressurizing) portion is denoted as 51. As the arterial blood vessels are anelastic, they experience small load/unload cycles as the various pulse waves of the waveform arrive, as denoted by 52. The three (3) component thick wall anelastic power law model denoted as 22, with inner wall radius 23 and outer wall radius 24, is fitted to the normalized arterial pulse pressure versus normalized arterial pulse volume for the twenty (20) subset of the forty (40) normotensive subjects, subjected to the effects of vasodilation due to NTG being administered. The rise (pressurizing) portion of the pulse pressure versus pulse volume for the power law model fitted to the measured data, is shown as 53, with a power law model value of β_{S}=1.25, and the purely fall (depressurizing) portion is denoted as 54, with a power law model value of β_{D}=0.4. The Quality factor, Q, for the fitted model shown as 49 in FIG. 11 is Q=6.5, which translates to a 15% energy loss over a complete load/unload cycle, significantly different to the forty (40) normotensive subjects having a Q=3.1. The Quality Factor of Q=6.5 is considered representative of healthy arterial vascular blood vessels, subject to significant vasodilation.

Note the significant difference in the rise (pressurizing) portion of 50 compared to 19, shown in FIG. 4, for the normotensive subjects prior to NTG being administered. The β_{S} value of >1 in FIG. 11, leads to a blood vessel stiffening with pulse pressure, clearly resulting in a significant change in the anelastic response of the arterial vessels to pulse pressure, both loading and unloading, due to vasodilation. In this case of vasodilation, the pulse volume response leads the pulse pressure response up to near the peak pulse volume; whereas, in the normotensive and hypertensive subjects, the pulse pressure leads the pulse volume response with time, during the rise (pressurizing) portion of the arterial vessels. It is the significant changes in the arterial blood vessels anelastic behavior under vasodilation, that result in the observed large drops in normalized pulse volume and normalized pulse pressure during diastolic. The reflected waves are not removed by the vasodilation, but the forward waves including the first pulse wave require a significant larger pulse volume to achieve the same pulse pressure, i.e. when pressurizing up the path 50, compared to pressurizing up the path 19, as is the case for the normotensive subjects. Thus, any forward waves result in much lower induced pulse pressure for the dilated arteries, and their reflected components are also much reduced. In the depressurizing state, a small change in pulse volume results in a significant change in pulse pressure, i.e. following path 51 compared to 20, and thus accounts for the large changes seen in the diastolic phase.

Induced vasocontraction is analogous to a negative pressure applied to the inner wall of the arterial blood vessels, and thus unloads the vessels along the unloading path of the anelastic model. Thus, for a very small contraction pressure, a moderate contraction volume change is achieved, requiring a rise in internal pressure to overcome the vasocontraction. Further increase in pulse pressure follows the loading (pressurizing) path, similar to the hypertension subjects as denoted by the anelastic model as 31, and then on unloading (depressurizing) the path denoted as 32, as shown in FIG. 5. Significant vasocontraction results in a high Q value, thus giving rise to significant damping of the high frequency shear waves. The contracted arteries unload (depressurize) along the path denoted as 32, but the arterial pressure remaining, as mentioned earlier to overcome the vasocontraction effect, will only dissipate by arterial windkessel flow, and can be ˜20% of the maximum pulse pressure. This impact results in the fall to rise time ratio of the first pulse wave to be <1 for the case of vasocontraction, as the early rise in pulse pressure has no induced pulse volume change, and so the initial rise time of the first pulse wave will be longer than the fall time. Therefore, vasocontraction not only increases the diastolic arterial pressure quite significantly for a small applied contraction pressure, but also increases the pulse pressure, and combined, significantly raises the systolic arterial pressure.

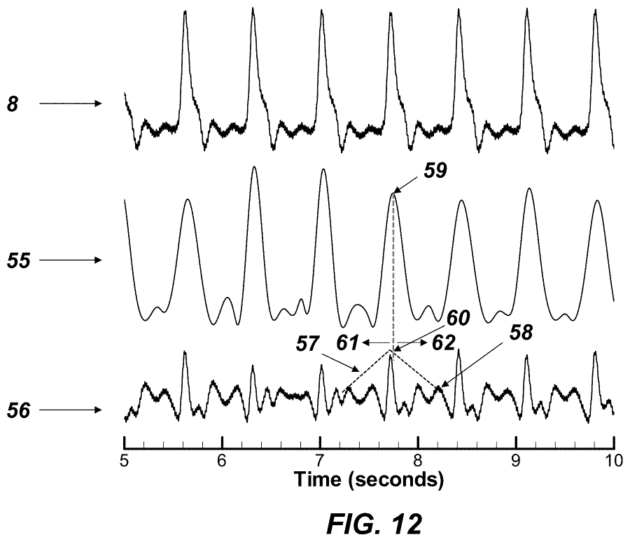

As depicted in FIG. 12, the graph illustrates an enlarged time history of the constructed arterial pulse optical plethysmograph waveform (dPVW) 8, and the recomposed EMD dPVW waveform 55, and the high frequency highly dissipative shear waveform mode 56. The high frequency highly dissipative waveform mode 56, is typical of the high frequency shear waves that are generated by the propagating arterial pressure pulse as a highly dissipative conical wake of high frequency shear waves. Similar behavior has been noted in the propagation of Stoneley waves in a slow medium in the geophysics literature, and for fluid filled boreholes, is known as the Scholte wave. The rise form of 56 denoted as 57 is dependent on the pulse waveform of 56, its propagating velocity and the properties of the blood and arterial blood vessels. The attenuation or decay of 56 as denoted by 58 is dependent on the material properties of the arterial blood vessels and the properties of the blood. The attenuation or decay can be computed via the logarithm decrement and the period of oscillation to yield the natural frequency and damping coefficient of the arterial blood vessels walls in the vicinity of the intravenous line inserted in the subject. These data can assess the state of the subject's arterial blood vessels and also quantify over time any change in the state of a subject's vasodilation, vasocontraction or hypertension.

$$Q = \frac{\sqrt{{π}^{2} + {δ}^{2}}}{2 δ} \qquad ( 3 )$$

Q is the Quality factor and δ is the logarithmic decrement, with Q and δ related as denoted in equation (3). The logarithmic decrement denoted by 58 of the waveform 56 is typically 0.51 for a healthy subject, being a Quality factor, Q=3.1. A subject with hypertension will imposed significant damping of these high frequency shear waves 56, as also would a subject undergoing vasocontraction. A normotensive subject subjected to vasodilation, will result in less damping of these shear waves. Thus, the extent of vasodilation and vasocontraction can be determined by the change in damping of these shear waves, i.e. a time phase shift of the two respective waveforms 55 and 56. Therefore, an alternative definition of the Vasodilation/Vasocontraction index VI is the change in damping of these high frequency shear waves. Such changes result in changes of time phase shift between the pulse waveform 55 shown at a peak time location as 59, with the high frequency shear waveform 56 with its peak time location shown as 60. The relative time phase shift between 59 and 60 depends on the degree of hypertension, and the extent of any vasodilation or vasocontraction. A vasocontraction or induced hypertension will time phase shift the waveform 56 peak 60 to an earlier 61 time location, compared to its relative time position with 59 prior to the vasocontraction, i.e. it will experience a time phase shift compared to its relative time location of the peak 59 of waveform 55, prior to the vasocontraction or induced hypertension. Similarly, a vasodilation will time phase shift the waveform 56 peak 60 to a later 62 time location, compared to its relative time position with 59 prior to the vasodilation, i.e. it will experience a time phase shift compared to its relative time location of the peak 59 of waveform 55. Depending on the measurement location and the subject's hypertensive state, for a normotensive subject, the time location of 60 may will be at a later 62 time position compared to 59, whereas, a hypertensive subject the peaks 59 and 60 will be closer and 60 will be at an earlier 61 relative time location compared to 59, i.e. the peak 60 can occur at an earlier time compared to the peak 59 of the waveform 55. The second forward pulse wave, which causes closure of the aortic valve, also generates a conical wake of high frequency highly dissipative shear waves, and if this pulse wave is significant due to hypertension or induced vasocontraction, it may destructively interfere with the shear waves generated by the first forward pulse wave. Such destructive wave interference of these high frequency shear waves quantifies the magnitude and phase of the interfering pulse wave, and also can determine the time location of the second forward pulse wave. Thus, it is important to consider the time location of 60, only in the cases of its time location being earlier than the second forward pulse wave, which causes aortic valve to close.

Sensed data from a pulse optical plethysmograph sensor placed over an artery, provides the measured waveform (PVW) and its first time derivative waveform (dPVW) is calculated and the high frequency highly dissipative conical wake of shear waves is removed from the dPVW waveform by the EMD or EEMD method, collectively denoted here as the EMD method. Similarly, a piezoelectric sensor could be placed over an artery can provide similar waveforms, such as the PAW, which is a direct rate of change waveform, and thus the EMD method can extract the high frequency highly dissipative conical wake of shear waves can be removed from the PAW waveform, and a new constructed form of the PAW is determined in order to quantify the damping of the shear waves, due to hypertension, or change during vasocontraction or vasodilation.

Finally, it will be understood that the preferred embodiment has been disclosed by way of example, and that other modifications may occur to those skilled in the art without departing from the scope and spirit of the appended claims.

What is claimed is:

1. A method of quantifying hypertension and aging status of a subject in near real time, the method comprising the steps of:
a. placing a pulse optical plethysmograph sensor adjacent to a blood vessel of a subject;
b. recording the pulse arterial volume waveform (PVW) from the sensor;
c. constructing a first time derivative waveform (dPVW) of the pulse arterial volume waveform (PVW);
d. determining the normalized ratio of the fall time to the rise time of the first pulse wave from the dPVW waveform;
e. computing the hypertensive index magnitude from this ratio;
f. displaying the hypertensive index; and
g. treating hypertension based on the hypertensive index.

2. The method of claim 1, wherein the pulse optical plethysmograph sensor is either an infra-red optical plethysmograph sensor, visible light optical plethysmograph sensor or pulse oximetry sensor.

3. The method of claim 1, wherein the subject's in vivo anelastic power law coefficients are computed and displayed.

4. The method of claim 1, wherein arrival time location of a second forward pulse wave on the PVW is determined, and from the second forward pulse wave on the PVW, the extent of the hypertension related to aging is determined and displayed.

5. The method of claim 1, wherein the extent of vasodilation or vasocontraction the blood vessel is determined from the normalized ratio of the fall time to rise time change of dPVW.

6. The method of claim 1, wherein the pulse optical plethysmograph sensor is placed over the finger.

7. The method of claim 1, wherein the pulse optical plethysmograph sensor is placed over an artery.

8. The method of claim 7, wherein the subject's in vivo anelastic power law coefficients and hypertrophy are computed and displayed.

9. The method of claim 4, wherein the PVW and its time derivatives are decomposed by the empirical mode decomposition method to quantify a normalized time shift.

10. The method of claim 9, wherein a high frequency conical wake of shear waves waveform is determined, and a damping and time phase shift of these shear waves is determined to quantify the time position of the second forward pulse wave, and the extent of vasocontraction, vasodilation or induced hypertension.

11. The method of claim 4, wherein the normalized ratio of change of pulse volume at the second forward pulse wave on the PVW waveform is determined, and the extent of vasodilation or vasocontraction of the subject's blood vessel is displayed.

12. The method of claim 11, wherein the PVW and its time derivatives are decomposed by the empirical mode decomposition method to quantify a normalized pulse volume ratio.

13. The method of claim 1, wherein a piezoelectric sensor is placed over an artery and its waveform recorded and arrival times between the piezoelectric sensor and the optical plethysmograph sensor are calculated and a pulse wave velocity is determined and displayed.

14. The method of claim 1, wherein the optical plethysmograph sensor is placed over an artery and its waveform recorded and arrival times between the optical plethysmograph sensor placed over the artery and an optical plethysmograph finger sensor are calculated and a pulse wave velocity is determined based on spacing between the optical plethysmograph sensor and the finger sensor, and the pulse wave velocity is displayed.

15. The method of claim 4, wherein a piezoelectric sensor is placed over an artery and its waveform recorded and the normalized time ratio of the second forward pulse wave is determined, for assessment of a normalized time shift to determine the extent of the hypertension related to aging.

16. The method of claim 1, wherein a piezoelectric sensor is placed over an artery and its waveform recorded and decomposed by the empirical mode decomposition method, and a high frequency conical wake of shear waves waveform is determined, the damping and time phase shift of these shear waves is determined to quantify the extent of vasocontraction, vasodilation or induced hypertension.

17. The method of claim 4, wherein a piezoelectric sensor is placed over an artery and its waveform recorded and the normalized time ratio of the second forward pulse wave is determined, for assessment of a normalized pulse volume ratio to be determined from a pulse volume rate of change (PAW) waveform, and the extent of vasodilation or vasocontraction of the artery is displayed.

18. The method of claim 17, the piezoelectric waveform is decomposed by the empirical mode decomposition method, wherein a high frequency conical wake of shear waves waveform is determined, a damping and time phase shift of these shear waves is determined to quantify the extent of vasocontraction, vasodilation or induced hypertension.

19. The method of claim 17, wherein the piezoelectric sensor placed over an artery, its waveform is integrated in the time vicinity of the second forward pulse wave to determine the pulse volume change, for assessment of the normalized pulse volume ratio, and the extent of vasodilation or vasocontraction of the subject is displayed.

20. The method of claim 19, wherein the piezoelectric waveform and its derivatives are decomposed by the empirical mode decomposition method to better quantify the normalized time ratio, for assessment of the normalized time shift to determine the extent of the hypertension related to aging.

21. The method of claim 18, wherein the decomposition, summing of intrinsic modes and display of normalized ratio is conducted on a sliding time window for the near real time display of the subject's vasodilation, vasocontraction or induced hypertension is displayed.

22. The method of claim 1, further comprising: making a determination, via an accelerometer of the computing device, that a current rate of movement of the subject is less than a threshold rate of movement, prior to performing steps (a)-(f).

23. A method comprising:
a. generating, via a sensor of a computing device, signals representing peripheral arterial pulse volume (PVW) waveforms originating from blood flowing through an anelastic blood vessel of a subject;
b. determining the first time derivative (dPVW) of the PVW waveforms;
c. determining the power law components of properties of the anelastic blood vessel and vasodilation/vasocontraction and hypertensive states of the blood vessels from the rise/fall time of the dPVW waveform;
d. determining arterial pulse wave velocity (PWV) from arrival times on the dPVW waveform; and
e. determining secant radial shear modulus and hypertrophy of the subject's blood vessels from the PVW and dPVW waveforms.

24. A method of claim 23, wherein the sensor comprises a pulse optical plethysmograph sensor or a piezoelectric sensor.

25. A method of any of claim 23, wherein the PVW waveform and a peripheral arterial pulse volume rate of change (PAW) waveform are generated by blood flowing through the subject's blood vessel.

26. The method of claim 24, wherein the sensors are positioned proximately to a peripheral artery, and wherein the waveforms originate from the peripheral artery.

27. The method of claim 26, wherein the subject is a human subject.

28. The method of claim 26, wherein the subject is breathing spontaneously while the signals are generated.

29. The method of claim 25, wherein anelastic power law coefficients, hypertrophy and Quality factor are determined from either the dPVW or PAW waveforms.

30. The method of claim 25, wherein a normalized time ratio is determined from empirical mode decomposition method of the PVW waveform and the PAW waveform.

31. The method of claim 25, wherein a normalized pulse volume ratio is determined from empirical mode decomposition method of the PVW waveform and the PAW waveform.

32. The method of claim 23, wherein a damping of a pulse excited wake of high frequency highly dispersive shear waves is determined from empirical mode decomposition method.

33. The method of claim 23, wherein the method comprises carrying out steps (a)-(f): (i) prior to carrying out a treatment of the subject; and (ii) after carrying out the treatment.

34. The method of claim 23, wherein the method comprises carrying out steps (a)-(f) continuously on the subject if the subject is suspected of sepsis.

35. The method of claim 23, further comprising providing, via a user interface of the computing device, an indication of one or more anelastic mechanical properties including hypertrophy.

36. The method of claim 35 further comprising: determining that the one or more anelastic mechanical properties indicate stiffening, plaque buildup, arteriosclerosis and/or elevated risk of aneurysm; and providing, via a user interface of the computing device, an indication that the anelastic mechanical properties indicates stiffening, plaque buildup, arteriosclerosis, and/or elevated risk of aneurysm or dissection.

37. The method of claim 29, further comprising: determining the Quality factor from the energy lost during a single pressure volume cardiac cycle; and using the determined Quality factor and anelastic mechanical properties to determine whether stiffening, plaque buildup, arteriosclerosis, elevated risk of aneurysm and/or other abnormal conditions are present in blood vessels of the subject; and providing, via a user interface of the computing device, an indication that the determined blood vessel properties indicates stiffening, plaque buildup, arteriosclerosis, and/or elevated risk of aneurysm or dissection.
